(19) United States
(12) Patent Application Publication
(10) Pub. No.: US 2026/0306657 A1
(43) Pub. Date: Oct. 8, 2026

(54) LIPOSOME CONJUGATE TARGETING M2 MACROPHAGE AND USE THEREOF

(71) Applicant: HANGZHOU NORMAL UNIVERSITY, Hangzhou (CN)

(72) Inventors: Tian XIE, Hangzhou (CN); Shuling WANG, Hangzhou (CN); Qingchang TIAN, Hangzhou (CN); Ruhua LUO, Hangzhou (CN); Xinyue LI, Hangzhou (CN)

(21) Appl. No.: 18/996,812

(22) PCT Filed: Jun. 28, 2023
(86) PCT No.: PCT/CN2023/102981

§ 371 Date: Jan. 17, 2025

(30) Foreign Application Priority Data
Jul. 28, 2022 (CN) 202210899297.4

(51) Int. Cl. A61K 47/69; A61K 31/7088; A61K 47/10; A61K 47/24; A61K 47/28; A61K 47/54; A61P 35/00

(52) U.S. Cl. CPC A61K 47/6911; A61K 31/7088; A61K 47/10; A61K 47/24; A61K 47/28; A61K 47/545; A61K 47/549; A61P 35/00

(57) ABSTRACT

A CpG liposome is surface-modified with folic acid (FA) and loaded with CpG, and 100 parts of the CpG liposome include, in parts by weight, 1 part to 5 parts of a phospholipid, 0.04 part to 0.2 parts of cholesterol, 0.004 parts to 0.1 parts of a poly(ethylene glycol) (PEG) derivative, 0.02 parts to 0.1 parts of a 1,2-distearoyl-sn-glycero-3-phosphoethanolamine-poly(ethylene glycol)-folic acid conjugate (DSPE-PEG-FA), 0.004 parts to 0.1 parts of the CpG, and water as a balance; and an ELE liposome is surface-modified with FA and AS1411 and loaded with elemene (ELE), and 100 parts of the ELE liposome include, in parts by weight, 1 part to 5 parts of the phospholipid, 0.04 parts to 0.2 parts of the cholesterol, 0.004 parts to 0.1 parts of the PEG derivative, 0.15 parts to 0.75 parts of the ELE, 0.02 parts to 0.1 parts of the DSPE-PEG-FA, 0.0005 parts to 0.002 parts of cholesterol-AS1411, and water as a balance. The combined use of the ELE liposome and the CpG liposome produces a synergistic effect of on tumor cells and can inhibit tumor growth and reverse immunosuppressive tumor microenvironment.

The present application claims priority to Chinese Patent Application No. CN202210899297.4 filed to the China National Intellectual Property Administration (CNIPA) on Jul. 28, 2022 and entitled “LIPOSOME CONJUGATE TARGETING M2 MACROPHAGE AND USE THEREOF”, which is incorporated herein by reference in its entirety.

### TECHNICAL FIELD

The present disclosure belongs to the technical field of pharmaceutical chemistry, especially the technical field of tumor chemotherapy drugs, and specifically relates to a liposome conjugate targeting M2 macrophages and use thereof.

### BACKGROUND

Breast cancer has long been considered a “cold” tumor with low immunogenicity. Due to the lack of infiltrating T cells and low permeability, breast cancer is one of the most difficult tumor types to be eliminated and is associated with poor patient prognosis. The breast cancer microenvironment is highly inflammatory. Inflammatory breast cancer patients highly express interleukin (IL)-8 and growth-regulated oncogene (GRO) chemokines, which activate signal transducer and activator of transcription 3 (STAT3), thus inducing the formation of immunosuppressive M2 macrophages and tumor mesenchymal cells, causing the cancer to become “cold”. It is a key issue to transform “cold” tumors into “hot” tumors and elucidate a complex positive feedback regulatory mechanism of the immune system.

Currently, treatments for breast cancer include chemotherapy, radiotherapy, surgery, and drug therapy. The difficulty in treating breast cancer so far is still the tumor metastasis, which is not only related to tumor spread, infiltration, and drug resistance, but also closely related to tumor angiogenesis, poor antigen presentation ability, and inhibitory tumor microenvironment. Nowadays, there are emerging treatments for breast cancer, such as immunotherapy, targeted therapy, and combined therapy such as novel adjuvant chemotherapy. Unlike traditional tumor therapies, the immunotherapy does not directly target tumor cells, but indirectly eliminates tumor cells by mobilizing immune response of the body.

Immunosuppressive tumor microenvironment (ITME) is the main cause of tumor resistance and refractoryness, and reversing the ITME is an important strategy to improve the efficacy of anti-tumor therapy. M2 tumor-associated macrophages (M2-TAMs) secrete a large number of immunosuppressive cytokines, inhibiting T cell immune responses and tumor infiltration, promoting tumor development and metastasis as well as angiogenesis, and shaping ITME to make the tumor “cool”. M1 macrophages secrete a large number of pro-inflammatory factors, recruiting and activating other immune cells, reversing the ITME, causing the tumor to “heat up”, and then improving treatment efficiency. Therefore, polarizing M2 macrophages in tumor tissues into M1 macrophages can be an important means to reverse ITME and improve anti-tumor effects.

An immune activator, cytosine-phosphate-guanine oligodeoxynucleotide (CpG-ODN) is an oligonucleotide sequence with immunostimulatory activity (sequence: TCCATGACGTTCCTGACGTT) that can effectively induce mammalian immune response. CpG-ODN cannot penetrate the cell membrane and is easily cleared by nucleases, and may induce inflammatory responses in the serum through systemic administration. Due to the lack of an effective administration route, the immune activator CpG is difficult to be delivered to M2 macrophages, limiting its in vivo application.

Elemene (ELE) extracted from the volatile oil of Curcuma wenyujin has a broad-spectrum anti-tumor activity, high efficiency, and low toxicity, can penetrate the blood-brain barrier (BBB), and shows an immune-protective effect in addition to the tumor-killing effect.

Nucleolin (NCL) is a multifunctional nucleolar phosphoprotein that plays an important role in cell proliferation, growth, and the occurrence of cytokines and nuclear biogenesis. The NCL is present in the nuclei of proliferating cells (normal and malignant) and also in the cytoplasm. In particular, NCL on the surface of tumor cells is conducive to the transport and cellular internalization of an aptamer AS1411. AS1411 is a synthetic quadruplex nuclease-resistant DNA oligonucleotide aptamer that induces proliferation arrest and apoptosis in a variety of cancer cells but has little effect on normal cells. The high affinity and specificity of AS1411 for NCL makes it a suitable targeting tool for functionalization of therapeutic drug payload delivery nanosystems to selectively target tumor cells. AS1411 can be used as an effective targeting molecule for targeting tumor cells. Compared with other targeting strategies, the AS1411 can target multiple types of tumors. Due to its non-immunogenic properties, this aptamer has proven to be safe in human trials and can improve the efficiency of drug delivery as an effective targeting ligand. In the near future, an aptamer AS1411-based nanodelivery system may become one of the effective strategies for cancer diagnosis and treatment for clinicians.

### SUMMARY

A first objective of the present disclosure is to provide a liposome conjugate targeting M2 macrophages, including a cytosine-phosphate-guanine (CpG) liposome and an elemene (ELE) liposome.

The CpG liposome is surface-modified with folic acid (FA) and loaded with an immune activator CpG, and every 100 parts of the CpG liposome include, in parts by weight, 1 part to 5 parts of phospholipid, 0.04 part to 0.2 parts of cholesterol, 0.004 parts to 0.1 parts of poly (ethylene glycol) derivative, (PEG) 0.02 parts to 0.1 parts of 1,2-distearoyl-sn-glycero-3-phosphoethanolamine-poly(ethylene glycol)-folic acid conjugate (DSPE-PEG-FA), 0.004 parts to 0.1 parts of immune activator CpG, and water as a balance.

the ELE liposome is surface-modified with FA and an aptamer AS1411 and loaded with ELE, and every 100 parts of ELE liposome include, in parts by weight, 1 part to 5 parts of phospholipid, 0.04 parts to 0.2 parts of cholesterol, 0.004 parts to 0.1 parts of PEG derivative, 0.15 parts to 0.75 parts of ELE, 0.02 parts to 0.1 parts of DSPE-PEG-FA, 0.0005 parts to 0.002 parts of cholesterol-AS1411, and water as a balance.

In the present disclosure, the phospholipid is one or more selected from the group consisting of a soybean phospholipid, a hydrogenated phospholipid, an egg yolk phospholipid, and a synthetic phospholipid.

Preferably, the PEG derivative is selected from the group consisting of D-a-tocopherol poly (ethylene glycol) succinate (TPGS) and DSPE-PEG; and

the DSPE-PEG-FA is one or more selected from the group consisting of DSPE-PEG1000-FA, DSPE-PEG2000-FA, and DSPE-PEG3000-FA.

A second objective of the present disclosure is to provide a method for preparing the liposome conjugate, including:
weighing the phospholipid, the cholesterol, the PEG derivative, and DSPE-PEG-FA in proportion and adding a resulting mixture into anhydrous ethanol, heating the resulting mixture at 50° C. to 60° C. until solids are dissolved, and then continuing to heat until the ethanol is completely removed to obtain a first organic phase I; and dissolving an immune activator CpG in double distilled water, adding the first organic phase I, and then conducting high-speed shearing and ultrasonication in sequence to obtain the CpG liposome; and
weighing the phospholipid, the cholesterol, the PEG derivative, and the DSPE-PEG-FA in proportion and adding a resulting mixture into anhydrous ethanol, heating the resulting mixture at 50° C. to 60° C. until solids are dissolved, continuing to heat until the ethanol is completely removed, and then adding the ELE in proportion to obtain a second organic phase II; and dissolving cholesterol-AS1411 in double distilled water, adding the second organic phase II, and then conducting high-speed shearing and ultrasonication in sequence to obtain the ELE liposome; where
the high-speed shearing is conducted at 8,000 r/min to 15,000 r/min for 20 min to 40 min; and the ultrasonication is conducted at 230 W to 240 W.

A third objective of the present disclosure is to provide use of the liposome conjugate or the liposome conjugate prepared by the preparation method in inhibiting tumor growth and reversing tumor immune microenvironment.

The present disclosure overcomes the problems of lack of effective administration routes and high toxicity when CpG is used to reverse the tumor microenvironment, and provides a liposome conjugate targeting M2 macrophages, including a CpG liposome and an ELE liposome. The CpG liposome can reverse the transformation of M2 macrophages into M1 macrophages in vivo and in vitro, reverse ITME, promote the infiltration of cytotoxic T cells, and effectively inhibit tumor growth. The ELE liposome can not only inhibit tumor cell proliferation, but also selectively inhibit M2 macrophages. The CpG liposome and the ELE liposome act synergistically on the M2 macrophages, improve utilization rate of the drug, and significantly enhance efficacy. Meanwhile, the present disclosure significantly reduces the toxicity and side effects of CpG and the irritation of ELE, significantly improves safety, and effectively avoids the side effects of the drug on normal brain cells and other tissues and organs. The combined use of the CpG liposome and the ELE liposome has a significant synergistic anti-tumor effect, reconstructs the tumor microenvironment, and provides a novel method for the treatment of breast cancer and the like.

### BRIEF DESCRIPTION OF THE DRAWINGS

To illustrate the examples of the present disclosure or the technical solutions in the prior art more clearly, the accompanying drawings required in the examples will be briefly introduced below.

FIG. 1 shows a scanning electron microscopy (SEM) image of the CpG/Lipo-FA prepared in Example 13 of the present disclosure;

FIG. 2 shows a SEM image of the ELE/Lipo-AS1411-FA prepared in Example 13 of the present disclosure;

FIG. 3 shows a schematic diagram for a particle size and a particle dispersity index (PDI) of the CpG/Lipo-FA at different sampling time periods;

FIG. 4 shows a schematic diagram for a particle size and a PDI of the ELE/Lipo-AS1411-FA at different sampling time periods;

FIG. 5 shows a schematic diagram for the cytotoxicity of free ELE, ELE/Lipo, ELE/Lipo-AS1411, and FA-ELE/Lipo-AS1411 at different concentrations on 4T1 cells; and

FIG. 6 shows a schematic diagram for the cytotoxicity of different concentrations of ELE on M1 and M2 macrophages.

### DETAILED DESCRIPTION OF THE EMBODIMENTS

The technical solutions of the present disclosure will be further described in detail below in conjunction with specific examples:

The content described in the examples of the specification is merely an enumeration of the implementations of the inventive conception, and the claimed scope of the present disclosure should not be construed as being limited to the specific forms stated in the examples. Equivalent technical means that come into the minds of those of skills in the art in accordance with the inventive conception also fall within the claimed scope of the present disclosure.

### Example 1

1 part of soybean phospholipid, 0.1 parts of cholesterol, 0.1 parts of TPGS, and 0.1 parts of DSPE-PEG1000-FA were dissolved in anhydrous ethanol, heated at 50° C. until the solids were dissolved, and then continued to heat until ethanol was completely removed to obtain a first organic phase I; 0.1 parts of CpG was dissolved in 98.6 parts of double distilled water, and then the first organic phase I was added to allow high-speed shearing and ultrasonication in sequence to obtain a CpG liposome; where the high-speed shearing was conducted at 8,000 r/min for 40 min, and the ultrasonication was conducted at 240 W.

### Example 2

1 part of soybean phospholipid, 1 part of egg yolk phospholipid, 0.2 parts of cholesterol, 0.04 parts of DSPE-PEG, 0.01 parts of DSPE-PEG1000-FA, and 0.01 parts of DSPE-PEG2000-FA were dissolved in anhydrous ethanol, heated at 52° C. until the solids were dissolved, and then continued to heat until ethanol was completely removed to obtain a first organic phase I; 0.04 parts of CpG was dissolved in 97.7 parts of double distilled water, and then the first organic phase I was added to allow high-speed shearing and ultrasonication in sequence to obtain a CpG liposome; where the high-speed shearing was conducted at 10,000 r/min for 30 min, and the ultrasonication was conducted at 230 W.

### Example 3

1 part of soybean phospholipid, 1 part of hydrogenated phospholipid, 1 part of synthetic phospholipid, 0.04 parts of cholesterol, 0.008 parts of TPGS, and 0.044 parts of DSPE-PEG3000-FA were dissolved in anhydrous ethanol, heated at 55° C. until the solids were dissolved, and then continued to heat until ethanol was completely removed to obtain a first organic phase I; 0.008 parts of CpG was dissolved in 96.9 parts of double distilled water, and then the first organic phase I was added to allow high-speed shearing and ultrasonication in sequence to obtain a CpG liposome; where the high-speed shearing was conducted at 12,000 r/min for 25 min, and the ultrasonication was conducted at 235 W.

### Example 4

4 parts of hydrogenated phospholipid, 0.06 parts of cholesterol, 0.004 parts of DSPE-PEG, and 0.032 parts of DSPE-PEG2000-FA were dissolved in anhydrous ethanol, heated at 56° C. until the solids were dissolved, and then continued to heat until ethanol was completely removed to obtain a first organic phase I; 0.004 parts of CpG was dissolved in 95.9 parts of double distilled water, and then the first organic phase I was added to allow high-speed shearing and ultrasonication in sequence to obtain a CpG liposome; where the high-speed shearing was conducted at 15,000 r/min for 20 min, and the ultrasonication was conducted at 238 W.

### Example 5

5 parts of egg yolk phospholipid, 0.15 parts of cholesterol, 0.06 parts of TPGS, 0.01 parts of DSPE-PEG1000-FA, 0.01 parts of DSPE-PEG2000-FA, and 0.01 parts of DSPE-PEG3000-FA were dissolved in anhydrous ethanol, heated at 60° C. until the solids were dissolved, and then continued to heat until ethanol was completely removed to obtain a first organic phase I; 0.06 parts of CpG was dissolved in 94.7 parts of double distilled water, and then the first organic phase I was added to allow high-speed shearing and ultrasonication in sequence to obtain a CpG liposome; where the high-speed shearing was conducted at 10,000 r/min for 35 min, and the ultrasonication was conducted at 240 W.

### Example 6

3 parts of synthetic phospholipid, 0.12 parts of cholesterol, 0.01 parts of DSPE-PEG, 0.03 parts of DSPE-PEG1000-FA, and 0.03 parts of DSPE-PEG3000-FA were dissolved in anhydrous ethanol, heated at 54° C. until the solids were dissolved, and then continued to heat until ethanol was completely removed to obtain a first organic phase I; 0.01 parts of CpG was dissolved in 96.8 parts of double distilled water, and then the first organic phase I was added to allow high-speed shearing and ultrasonication in sequence to obtain a CpG liposome; where the high-speed shearing was conducted at 9,000 r/min for 40 min, and the ultrasonication was conducted at 230 W.

### Example 7

1 part of soybean phospholipid, 0.1 parts of cholesterol, 0.1 parts of TPGS, and 0.1 parts of DSPE-PEG1000-FA were dissolved in anhydrous ethanol, heated at 50° C. until the solids were dissolved, continued to heat until ethanol was completely removed, and then 0.3495 parts of ELE was added to obtain a second organic phase II; 0.0005 parts of cholesterol-AS1411 was dissolved in 98.35 parts of double distilled water, and then the first organic phase II was added to allow high-speed shearing and ultrasonication in sequence to obtain an ELE liposome; where the high-speed shearing was conducted at 8,000 r/min for 40 min, and the ultrasonication was conducted at 240 W.

### Example 8

1 part of soybean phospholipid, 1 part of egg yolk phospholipid, 0.2 parts of cholesterol, 0.04 parts of DSPE-PEG, 0.01 parts of DSPE-PEG1000-FA, and 0.01 parts of DSPE-PEG2000-FA were dissolved in anhydrous ethanol, heated at 52° C. until the solids were dissolved, continued to heat until ethanol was completely removed, and then 0.15 parts of ELE was added to obtain a second organic phase II; 0.001 parts of cholesterol-AS1411 was dissolved in 97.589 parts of double distilled water, and then the first organic phase II was added to allow high-speed shearing and ultrasonication in sequence to obtain an ELE liposome; where the high-speed shearing was conducted at 10,000 r/min for 30 min, and the ultrasonication was conducted at 230 W.

### Example 9

3 parts of synthetic phospholipid, 0.12 parts of cholesterol, 0.01 parts of DSPE-PEG, 0.03 parts of DSPE-PEG1000-FA, and 0.03 parts of DSPE-PEG3000-FA were dissolved in anhydrous ethanol, heated at 54° C. until the solids were dissolved, continued to heat until ethanol was completely removed, and then 0.75 parts of ELE was added to obtain a second organic phase II; 0.0015 parts of cholesterol-AS1411 was dissolved in 96.0585 parts of double distilled water, and then the first organic phase II was added to allow high-speed shearing and ultrasonication in sequence to obtain an ELE liposome; where the high-speed shearing was conducted at 9,000 r/min for 40 min, and the ultrasonication was conducted at 230 W.

### Example 10

4 parts of hydrogenated phospholipid, 0.06 parts of cholesterol, 0.004 parts of DSPE-PEG, and 0.032 parts of DSPE-PEG2000-FA were dissolved in anhydrous ethanol, heated at 56° C. until the solids were dissolved, continued to heat until ethanol was completely removed, and then 0.402 parts of ELE was added to obtain a second organic phase II; 0.002 parts of cholesterol-AS1411 was dissolved in 95.5 parts of double distilled water, and then the first organic phase II was added to allow high-speed shearing and ultrasonication in sequence to obtain an ELE liposome; where the high-speed shearing was conducted at 15,000 r/min for 20 min, and the ultrasonication was conducted at 238 W.

### Example 11

1 part of soybean phospholipid, 1 part of hydrogenated phospholipid, 1 part of synthetic phospholipid, 0.04 parts of cholesterol, 0.008 parts of TPGS, and 0.044 parts of DSPE-PEG3000-FA were dissolved in anhydrous ethanol, heated at 55° C. until the solids were dissolved, continued to heat until ethanol was completely removed, and then 0.507 parts of ELE was added to obtain a second organic phase II; 0.001 parts of cholesterol-AS1411 was dissolved in 96.4 parts of double distilled water, and then the first organic phase II was added to allow high-speed shearing and ultrasonication in sequence to obtain an ELE liposome; where the high-speed shearing was conducted at 12,000 r/min for 25 min, and the ultrasonication was conducted at 235 W.

### Example 12

5 parts of egg yolk phospholipid, 0.15 parts of cholesterol, 0.06 parts of TPGS, 0.01 parts of DSPE-PEG1000-FA, 0.01 parts of DSPE-PEG2000-FA, and 0.01 parts of DSPE-PEG3000-FA were dissolved in anhydrous ethanol, heated at 60° C. until the solids were dissolved, continued to heat until ethanol was completely removed, and then 0.6592 parts of ELE was added to obtain a second organic phase II; 0.0008 parts of cholesterol-AS1411 was dissolved in 94.1 parts of double distilled water, and then the first organic phase II was added to allow high-speed shearing and ultrasonication in sequence to obtain an ELE liposome; where the high-speed shearing was conducted at 10,000 r/min for 35 min, and the ultrasonication was conducted at 240 W.

### Example 13

Based on a total weight of 100 g, the CpG liposome (CpG/Lipo-FA) included: 2 g of soybean phospholipid, 0.1 g of cholesterol, 0.004 g of DSPE-PEG2000, 0.04 g of CpG, 0.04 g of DSPE-PEG2000-FA, and water as a balance.

Based on a total weight of 100 g, the ELE liposome (ELE/Lipo-AS1411-FA) included: 2 g of soybean phospholipid, 0.1 g of cholesterol, 0.004 g of DSPE-PEG2000, 0.25 g of ELE, 0.04 g of DSPE-PEG2000-FA, 0.002 g of cholesterol-AS1411, and water as a balance.

### a Preparation Method of the CpG/Lipo-FA Included:

(1) Preparation of an organic phase: the phospholipid, cholesterol, PEG derivative, and DSPE-PEG-FA were weighed in proportion and dissolved in ethanol, heated at 60° C. until dissolved, and then continued to heat until ethanol was completely removed to obtain an organic phase I.

(2) Preparation of an aqueous phase: the CpG was weighed and dissolved in double distilled water to obtain an aqueous phase I.

(3) Ultrasonic dispersion: the organic phase I was added into the aqueous phase I and then subjected to high-speed shearing and ultrasonication in sequence to obtain the CpG/Lipo-FA.

### a Preparation Method of the ELE/Lipo-AS1411-FA Included:

(1) Preparation of an organic phase: the phospholipid, cholesterol, PEG derivative, and DSPE-PEG-FA were weighed in proportion and dissolved in ethanol, heated at 60° C. until dissolved, and continued to heat until ethanol was completely removed to obtain an organic phase I; the ELE was weighed in proportion and dissolved in the organic phase I to obtain an organic phase II.

(2) Preparation of an aqueous phase: the cholesterol-AS1411 was weighed and dissolved in double distilled water to obtain an aqueous phase II.

(3) Ultrasonic dispersion: the organic phase II was added into the aqueous phase II and then subjected to high-speed shearing and ultrasonication in sequence to obtain the ELE/Lipo-AS1411-FA.

### 1. Sem Characterization:

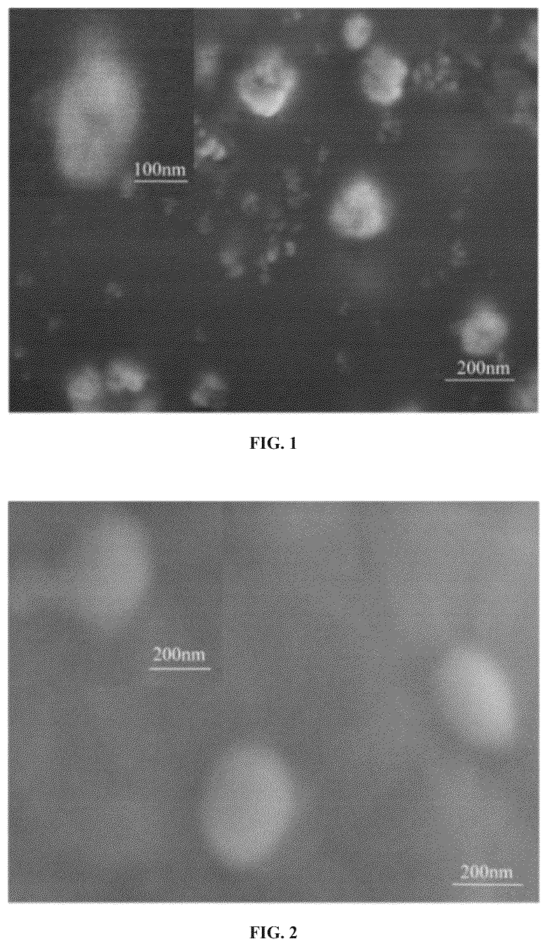

The newly prepared liposome solution was diluted to a concentration of 0.01 mg/mL, and a drop of about 10 μL of the diluted liposome solution was dripped on a silicon wafer of appropriate size. The solution was slowly blown flat with nitrogen to make it evenly dispersed and accelerate drying. After drying, it was sprayed with gold and then the microscopic morphology of the liposome was observed by SEM. The results are shown in FIG. 1 and FIG. 2, where FIG. 1 shows a SEM image of CpG/Lipo-FA prepared in this example, and FIG. 2 shows a SEM image of ELE/Lipo-AS1411-FA. SEM observation was the morphology observation of the liposome after drying. Due to loss of water in the particles, the liposome particle size measured by SEM might be slightly smaller than the result measured by the particle size analyzer.

### 2. Determination of Particle Size, Particle Dispersity Index (PDI), and Zeta Potential:

According to the method of this example, liposomes without DSPE-PEG-FA, cholesterol-AS1411, and DSPE-PEG-FA (ELE/Lipo and ELE/Lipo-AS1411) were prepared.

The CpG/Lipo-FA, ELE/Lipo-AS1411-FA prepared in this example and the above-mentioned ELE/Lipo, ELE/Lipo-AS1411 were taken and the particle size and PDI of the liposomes were measured using a nanoparticle sizer. 50 μL of liposomes were placed in an absorption pool of the particle sizer, and the particle size, PDI, and Zeta potential were measured after dilution according to a certain ratio, and the results are shown in Table 1.

Table 1 Determination results of particle size, PDI, and Zeta potential for different liposomes (n=3; mean±SD)

| Item | Size (nm) ± SD | PDI ± SD | ZP (mV) ± SD |
|---|---|---|---|
| ELE/Lipo | 118.0 ± 2.715 | 0.1467 ± 0.0046 | 20.90 ± 1.443 |
| ELE/Lipo- | 105.0 ± 0.7506 | 0.0997 ± 0.0131 | 23.74 ± 1.850 |
| AS1411 | | | |
| ELE/Lipo- | 144.0 ± 3.439 | 0.0337 ± 0.0285 | 23.41 ± 2.227 |
| AS1411-FA | | | |
| CpG/Lipo-FA | 158.4 ± 3.568 | 0.1990 ± 0.0062 | 24.55 ± 1.323 |

PDI is an important indicator to measure the uniformity of liposome particle size, and the lower the PDI is, the more uniform the particle dispersion can be. Table 1 shows that the ELE/Lipo-AS1411 and ELE/Lipo-AS1411-FA prepared by the preparation method had moderate particle size and uniform particle size distribution.

### 3. Encapsulation Efficiency and Drug Loading Capacity:

0.2 mL of each of the CpG/Lipo-FA, ELE/Lipo-AS1411-FA prepared in this example and the above-mentioned ELE/Lipo and ELE/Lipo-AS1411 were added into a 0.45 μm PVDF microfiltration centrifuge tube and centrifuged at 20° C. and 12,000 rpm for 10 min to retain the ELE droplets that were not loaded into the liposomes. Into the lower layer liquid was added 25 mL of acetonitrile, and the content of the drug in the lower layer was detected as A. Acetonitrile was added into an upper layer liquid to make it 25 mL, ultrasonicated for 30 min, and the content of drug B in the upper layer was detected as B. After passing through the membrane, high-performance liquid chromatography (HPLC) was conducted for detection. The amount of auxiliary materials and drugs added when preparing the liposome was Z. At the same time, the encapsulation efficiency of the CpG/Lipo-FA was determined by NanoDrop2000. The encapsulation efficiency and drug loading capacity were calculated: encapsulation efficiency (EE)=A/(A+B)×100%, drug loading capacity (DLC)=A/Z×100%.

The drug loading capacity and encapsulation efficiency of the ELE-encapsulated liposomes were tested by HPLC, and the results are shown in Table 2.

TABLE 2
Test results of drug loading capacity and encapsulation efficiency
of different liposomes (n = 3; mean ± SD)

| Item | Size (nm) ± SD | PDI ± SD |
|---|---|---|
| ELE/Lipo | 93.31 ± 0.2566 | 17.74 ± 0.04583 |
| ELE/Lipo-AS1411 | 98.63 ± 0.1137 | 18.90 ± 0.01528 |
| ELE/Lipo-AS1411-FA | 95.37 ± 1.1000 | 19.17 ± 0.21940 |
| CpG/Lipo-FA | 74.07 ± 0.9018 | N/A |

Table 2 shows that the encapsulation efficiency of ELE in the three ELE-encapsulated liposomes basically reached not less than 90%, and the relevant targeting molecules had no obvious effect on their encapsulation efficiency, and all showed a high drug loading capacity. The encapsulation efficiency of CpG/Lipo-FA was (74.07±0.9018) %.

### 4. Liposome Stability Test

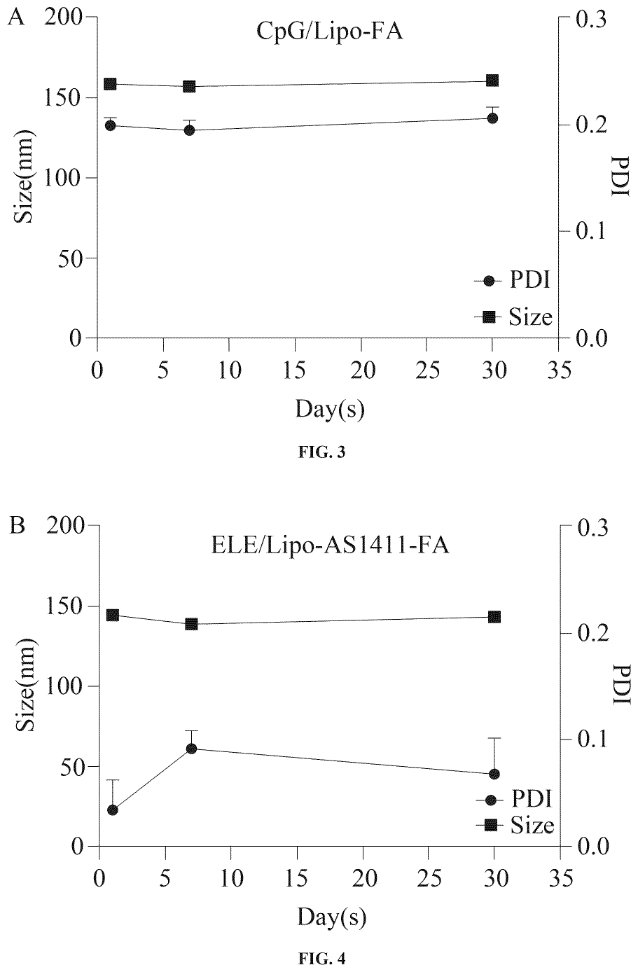

The ELE/Lipo-AS1411 and ELE/Lipo-AS1411-FA prepared in this example were stored at 4° C., and samples were taken at different times of day, day 7, and day 30, respectively. The particle size, PDI, and Zeta potential of the liposomes prepared at different time periods were measured by a particle size analyzer. FIG. 3 and FIG. 4 show that the particle size and PDI of the two liposomes fluctuated within a small range within 30 days, and their appearance did not change significantly, and no precipitation or stratification occurred, showing desirable stability.

### Example 14

### Investigation of Liposome Targeting Ability:

According to the method of Example 13, an FITC fluorescent marker was added during the liposome preparation to prepare Lipo-FITC, Lipo-FA-FITC, and AS1411-Lipo-FA-FITC.

### (1) Targeting Research of Nano-Delivery System for M2 Macrophages:

In order to investigate the M2 macrophage-specific targeting ability of the prepared nanoliposomes, macrophage uptake experiments were conducted using liposomes labeled with FITC fluorescence and having the same structure as the targeting molecule.

The results showed that when IL-4 was added to induce RAW264.7 cells into M2 macrophages, Lipo-FITC and Lipo-FA-FITC were added to incubate with M2 macrophages. A small amount of green fluorescence appeared in the non-induced RAW264.7 cells after incubation with Lipo-FA-FITC and in the IL-4+Lipo-FITC group. However, after incubation of Lipo-FA-FITC with M2 macrophages, the intracellular green fluorescence intensity increased significantly and was significantly different from the other two groups, that is, M2 macrophages took up less blank Lipo-FITC but could take up a large amount of Lipo-FA-FITC. This indicated that the Lipo-FA entered the cell through endocytosis mediated by FA receptors on the surface of M2 macrophages and was targeted to M2 macrophages.

### (2) Study on the Targeted Uptake of the Nano-Delivery System by 4T1 Tumor Cells:

### 1) Specific Binding of Aptamer AS1411 to 4T1 Tumor Cells:

4T1 cells were incubated with AS1411-FAM and C-AS1411-FAM (AS1411 as a control group, and C-AS1411 as a random nucleotide sequence) for 1 h, and the fluorescence intensity was observed under a confocal microscope. The results showed that the AS1411-FAM group had the highest fluorescence intensity, further proving that AS1411 could specifically target tumor cells rather than specifically adsorb nucleic acids.

### 2) Specific Binding of 4T1 Cells to Targeting Liposomes:

Nucleolin (NCL), which was highly expressed on the surface of 4T1 cells, could form a G-rich tetrad structure with the G-rich aptamer AS141, which could serve as a new tumor target. The results showed that after Lipo-FITC, Lipo-FA-FITC, and AS1411-Lipo-FA-FITC were co-incubated with 4T1 tumor cells, only a small amount of green fluorescence appeared in the cells of the Lipo-FITC and Lipo-FA-FITC groups, indicating that 4T1 tumor cells had little uptake of Lipo-FITC and Lipo-FITC. However, after AS1411-Lipo-FA-FITC was incubated with 4T1 cells, a large amount of green fluorescence was observed in the cells, indicating that AS1411-Lipo-FA-FITC could be taken up by 4T1 tumor cells in large quantities. In addition, the fluorescence intensity of AS1411-Lipo-FA-FITC was significantly higher than that of Lipo-FA-FITC, indicating that the simultaneous targeted modification of AS1411 and FA could significantly increase the targeted uptake of liposomes by tumor cells and showed targeting to tumor cells.

### Example 15

Study on the Ability of CpG Liposomes to Polarize M2 Macrophages into M1 Macrophages:

Real-time PCR was conducted to detect the changes in the transcription levels of M1 and M2 marker genes in RAW264.7 to verify the ability of CpG liposomes to polarize M2 macrophages into M1 macrophages. RAW264.7 cells were induced into M2 macrophages by IL-4 (20 ng/ml), and then treated with PBS, CpG/Lipo, and CpG/Lipo-FA. After the cells were collected, RNA was extracted and reverse-transcribed into cDNA, and Real-time PCR was conducted to detect changes in the mRNA levels of RAW264.7-related genes. Taking iNOS mRNA as an example, after IL-4 induction, iNOS mRNA was significantly up-regulated to 1.620-0.098 and 1.697=0.2558 times of the control group after adding CpG/Lipo and CpG/Lipo-FA, and the up-regulation of CpG/Lipo-FA was more significant and statistically different (vs.IL-4, *** P<0.001); while Arg-1 mRNA was significantly reduced to 1.295±0.1061 and 0.5733±0.4343 times of the control group, and the down-regulation of CpG/Lipo-FA was more significant and statistically different (vs.IL-4, ** P<0.01). The above results proved that CpG/Lipo-FA more significantly polarized M2 macrophages into M1 macrophages, and further proved that FA could target M2 macrophages.

Double immunofluorescence was adopted to verify the ability of CpG/Lipo-FA to reverse-polarized M2 macrophages. M2 macrophages induced by IL-4 had high expression of related protein Arg-1, but low expression of iNOS. After treating M2 macrophages with CpG/Lipo and CpG/Lipo-FA, the expression of Arg-1, a marker of M2 macrophages, decreased, and its fluorescence intensity showed a significant decrease, while the expression of iNOS, a marker of M1 macrophages, increased, indicating that both CpG/Lipo and CpG/Lipo-FA could polarize M2 macrophages to M1. Moreover, CpG/Lipo-FA was superior to CpG/Lipo, indicating that CpG could significantly reverse M2 macrophages into M1 macrophages, and CpG/Lipo-FA had the strongest effect in polarizing M2 macrophages into M1 macrophages.

### Example 16

### Cytotoxicity Studies of Tumor-Targeted Liposomes:

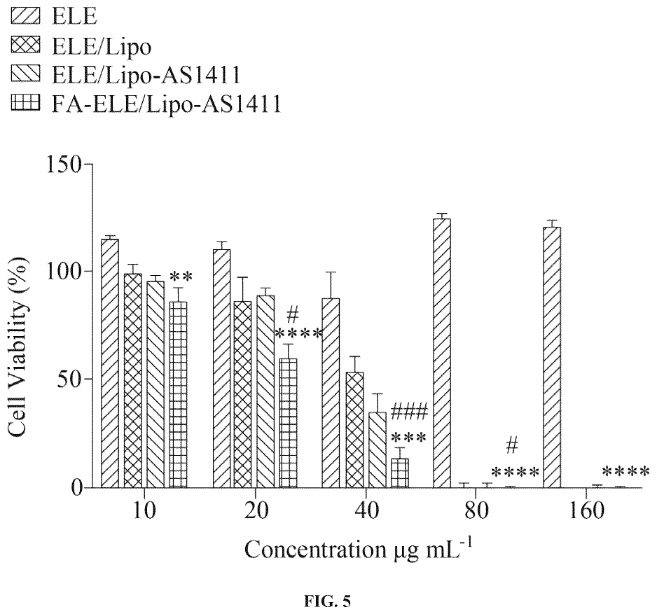

(1) The toxicity of ELE to 4T1 breast cancer cells was studied using a CCK-8 method. 4T1 cells (2×10 4 cells/well) were inoculated in a 96-well plate. After culturing for 24 h, the cells were treated with RMPI-1640 media containing ELE, ELE/Lipo, ELE/Lipo-AS1411, and FA-ELE/Lipo-AS1411 (drug concentrations being 10 μg/mL, 20 μg/mL, 40 μg/mL, 80 μg/mL, and 160 μg/mL, respectively) for 24 h. ELE was dissolved and diluted in medium containing 1% 0 ethanol, and untreated cells were used as a control. 100 μL of 10% CCK-8 solution was added into each well and incubated for 2 h. The absorbance at 450 nm was measured using an enzyme-labeled instrument, and the IC₅₀ value of each drug group was calculated and analyzed using Graphpad9.0. A schematic diagram of the cytotoxicity of different concentrations of free ELE, ELE/Lipo, ELE/Lipo-AS1411, and FA-ELE/Lipo-AS1411 on 4T1 cells was shown in FIG. 5, where n=3, x±SD, ** P<0.01, *** P<0.001, **** P<0.0001 (vs. ELE); #P<0.05, ###P<0.001 (vs. ELE/Lipo-AS1411).

As shown in FIG. 5, after the cells were acted on by each drug for 24 h, the IC₅₀ values were 921.4 μg/mL, 67.58 μg/mL, 44.78 μg/mL, and 35.02 μg/mL, respectively. The results showed that FA-ELE/Lipo-AS1411 had significant cytotoxicity to 4T1 cells at 4 concentrations, with significant differences; FA-ELE/Lipo-AS1411 could significantly reduce the survival rate of cells at concentrations of 20 μg/mL, 40 μg/mL, and 80 μg/mL (vs. ELE/Lipo-AS1411, * P<0.001), confirming that FA-ELE/Lipo-AS1411 could enhance the toxic effect of ELE on tumor cells.

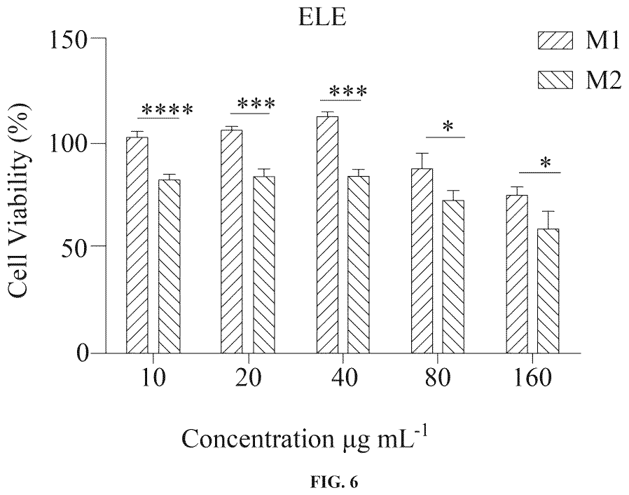

(2) The toxicity of ELE on M1 and M2 macrophages was studied using the CCK-8 method. RAW264.7 cells were pre-treated to polarize into M1 and M2 macrophages, and the M1 and M2 macrophages (2×10⁵ cells/well) were inoculated in a 96-well plate. After culturing for 24 h, the cells were treated with 100 μL of DMEM high-glucose media containing ELE (drug concentrations being 10 μg/mL, 20 μg/mL, 40 μg/mL, 80 μg/mL, and 160 μg/mL, respectively) for 24 h. ELE was dissolved and diluted in medium containing 1% % ethanol, and untreated cells were used as a control. 100 μL of 10% CCK-8 solution was added into each well and incubated for 2 h. The absorbance at 450 nm was measured using an enzyme-labeled instrument, and the IC 50 values of each group of drugs were calculated and analyzed using Graphpad Prism 9.0. A schematic diagram of the cytotoxicity of different concentrations of ELE on M1 and M2 macrophages is shown in FIG. 6, where n=3, x±SD, * P<0.05, *** P<0.001, **** P<0.0001.

According to FIG. 6, ELE had a selective toxic effect on M2 macrophages at 5 concentrations, and the results were statistically significant (all below * P<0.05). This indirectly proved that the ELE and CpG/Lipo-FA could jointly reverse and inhibit tumor suppressive M2 macrophages.

### Example 17 Study on the In Vivo Anti-Tumor Efficacy of M2 Macrophage-Targeted Liposomes

4T1 breast cancer cells in a logarithmic growth phase were digested and collected, centrifuged, resuspended in PBS and cell density-adjusted, and then placed in an ice box for later use. Female BALB/c mice (female, 4-6 weeks old, SPF-grade) were bred for 1 week, anesthetized, a tumor site to be inoculated was depilated and disinfected, and then 100 μL of a resulting cell suspension above was injected subcutaneously at the site.

(1) Anti-tumor efficacy of CpG/Lipo-FA: the day of tumor inoculation was designated as day 0. On day 8 after inoculation, the mice were divided into 4 groups, with 4 mice in each group, namely, a Saline group, a Lipo group, a CpG/Lipo group, and a CpG/Lipo-FA group. The CpG was administered with a dose of 4 μg per mouse, all of which were administered by intratumoral injection. The mice were weighed every other day using an electronic balance during the dosing period, and a maximum diameter of the tumor recorded as A (mm) and a minimum diameter recorded as B (mm) were measured with a vernier caliper. The mouse tumor volume V (mm³) was calculated according to V=A×B²/2, and the tumor was removed and weighed every other day after the last dose.

The results showed that the model control group grew quickly, and the tumor volume increased rapidly on day 14 after tumor inoculation. On the second day after the end of administration, compared with the Saline group, the tumor volume and tumor weight of the CpG/Lipo-FA group were significantly reduced, indicating a significant anti-tumor therapeutic effect, and there was no significant effect on the body weight of the mice, indicating no significant toxic effect.

(2) Antitumor efficacy of CpG/Lipo-FA combined with AS1411-ELE/Lipo-FA: the day of tumor inoculation was designated as day 0. On day 8 after inoculation, the mice were divided into 6 groups, with 4 mice in each group, namely a Saline group, a Lipo group, a CpG/Lipo-FA group, an AS1411-ELE/Lipo-FA group, an AS1411-ELE/Lipo-FA (7d)+CpG/Lipo-FA (7d) group (i.e., AS1411-ELE/Lipo-FA was injected for seven consecutive days, followed by CpG/Lipo-FA for seven days, referred to as Scheme A), and an AS1411-ELE/Lipo-FA (1d)+CpG/Lipo-FA (1d) group (i.e., AS1411-ELE/Lipo-FA was injected for one day, followed by CpG/Lipo-FA for one day, for a total of 7 times, referred to as Scheme B). The ELE dosage was 50 mg/Kg (intraperitoneal injection), and the CpG dosage was 4 μg per mouse (intratumoral injection). The mice were weighed on alternate days with an electronic balance, and a maximum diameter of the tumor recorded as A (mm) and a minimum diameter recorded as B (mm) were measured with a vernier caliper. The mouse tumor volume V (mm³) was calculated according to the formula V=A×B 2/2. At the same time, the tumor inhibition rate of each treatment group was calculated, tumor inhibition rate (%)=(V control−V)/V control×100%.

The results showed that the tumor in the model group mice grew rapidly, entering a rapid growth phase around 12 d after inoculation, with the tumor volume growing rapidly, and growing to about 1,000 mm³ on the day 14. Compared with the model group, the CpG/Lipo-FA group and AS1411-ELE/Lipo-FA group showed certain anti-tumor effects, with tumor inhibition rates of 52.58% and 61.13%, respectively. The anti-tumor effect of ELE liposome and CpG liposome was investigated by combining them with different dosing intervals. The results showed that after the end of treatment, the AS1411-ELE/Lipo-FA (7d)+CpG/Lipo-FA (7d) (A) group and the AS1411-ELE/Lipo-FA (1d)+CpG/Lipo-FA (1d) (B) group also showed anti-tumor properties, with tumor inhibition rates of 52.55% and 75.24%, respectively, indicating that the effect of Scheme B was more significant. However, there was a significant difference in the tumor inhibition rate, which might be related to the anti-inflammatory effect of ELE. Although continuous injection of ELE for 7 d could achieve the effect of tumor treatment, CpG/Lipo-FA could not reverse the anti-inflammatory effect of ELE. However, the results showed that alternating administration of the two drugs could enhance the tumor treatment effect, and further verified that CpG/Lipo-FA (4 μg/per) could reverse ITME caused by intermittent injection of ELE.

In conclusion, the combined use of ELE liposome and CpG liposome has a better efficacy against breast cancer. Moreover, the synergistic effect of ELE liposome and CpG liposome on tumor cells can inhibit tumor growth and reverse ITME, thereby enhancing the therapeutic effect, significantly reducing the side effects of CpG and the irritation of ELE, improving safety, and then exhibiting desirable clinical translation application prospects for breast cancer.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

1. A liposome conjugate targeting M2 macrophages, comprising a cytosine-phosphate-guanine (CpG) liposome and an elemene (ELE) liposome; wherein
the CpG liposome is surface-modified with folic acid (FA) and loaded with an immune activator CpG, and 100 parts of the CpG liposome comprise, in parts by weight, 1 part to 5 parts of phospholipid, 0.04 part to 0.2 parts of cholesterol, 0.004 parts to 0.1 parts of poly(ethylene glycol) (PEG) derivative, 0.02 parts to 0.1 parts of 1,2-distearoyl-sn-glycero-3-phosphoethanolamine-poly(ethylene glycol)-folic acid conjugate (DSPE-PEG-FA), 0.004 parts to 0.1 parts of immune activator CpG, and balanced water; and
the ELE liposome is surface-modified with FA and an aptamer AS1411 and loaded with ELE, and 100 parts of the ELE liposome comprise, in parts by weight, 1 part to 5 parts of phospholipid, 0.04 parts to 0.2 parts of cholesterol, 0.004 parts to 0.1 parts of PEG derivative, 0.15 parts to 0.75 parts of ELE, 0.02 parts to 0.1 parts of DSPE-PEG-FA, 0.0005 parts to 0.002 parts of cholesterol-AS1411, and balance water.

2. The liposome conjugate according to claim 1, wherein the phospholipid is one or more selected from the group consisting of a soybean phospholipid, a hydrogenated phospholipid, an egg yolk phospholipid, and a synthetic phospholipid.

3. The liposome conjugate according to claim 1, wherein the PEG derivative is selected from the group consisting of D-a-tocopherol poly(ethylene glycol) succinate (TPGS) and DSPE-PEG.

4. The liposome conjugate according to claim 1, wherein the DSPE-PEG-FA is one or more selected from the group consisting of DSPE-PEG1000-FA, DSPE-PEG2000-FA, and DSPE-PEG3000-FA.

5. The liposome conjugate according to claim 1, wherein the CpG liposome is prepared by a method comprising: weighing the phospholipid, the cholesterol, the PEG derivative, and the DSPE-PEG-FA in proportion and adding a resulting mixture into anhydrous ethanol, heating the resulting mixture at 50° C. to 60° C. until solids were dissolved, and then continuing to heat until ethanol is completely removed to obtain a first organic phase I; and dissolving the immune activator CpG in double distilled water, adding the first organic phase I, and then conducting high-speed shearing and ultrasonication in sequence to obtain the CpG liposome.

6. The liposome conjugate according to claim 1, wherein the ELE liposome is prepared by a method comprising: weighing the phospholipid, the cholesterol, the PEG derivative, and the DSPE-PEG-FA in proportion and adding a resulting mixture into anhydrous ethanol, heating the resulting mixture at 50° C. to 60° C. until solids dissolved, continuing to heat until ethanol is completely removed, and then adding the ELE in proportion to obtain a second organic phase II; and dissolving the cholesterol-AS1411 in double distilled water, adding the second organic phase II, and then conducting high-speed shearing and ultrasonication in sequence to obtain the ELE liposome.

7. The liposome conjugate according to claim 5, wherein the high-speed shearing is conducted at 8,000 r/min to 15,000 r/min for 20 min to 40 min.

8. The liposome conjugate according to claim 5, wherein the ultrasonication is conducted at 230 W to 240 W.

9-11. (canceled)

12. A method for inhibiting tumor growth and reversing tumor immune microenvironment, comprising: administering the ELE liposome and the CpG liposome in the liposome conjugate according to claim 1 to a subject in need thereof.

13. The method, according to claim 12, comprising:
injecting the ELE liposome in the liposome conjugate according to claim 1 for seven consecutive days, and then continuously injecting the CpG liposome in the liposome conjugate according to any one of claims 1 to 8 or a liposome conjugate prepared by the preparation method according to claim 9 for seven days; wherein
the ELE liposome is injected intraperitoneally with an injection dose of 50 mg/kg mouse; and
the CpG liposome is injected intratumorally with an injection dose of 50 μg/mouse.

14. The method according to claim 12, comprising:
alternately administering the ELE liposome and the CpG liposome in the liposome conjugate according to claim 1 to a subject in need thereof, wherein the ELE liposome is injected for one day and then the CpG liposome is injected for another day, and the ELE liposome and the CpG liposome each are injected for a total of seven days;
the ELE liposome is injected intraperitoneally with an injection dose of 50 mg/kg mouse; and
the CpG liposome is injected intratumorally with an injection dose of 50 μg/mouse.


SEQUENCE LISTING (1 sequence)

SEQ ID NO: 1 - DNA, 20 bp, synthetic construct
  source 1..20: mol_type = other DNA
tccatgacgttcctgacgtt